(19) United States
(12) Patent Application Publication
(10) Pub. No.: US 2023/0045171 A1
(43) Pub. Date: Feb. 9, 2023

(54) ADENO-ASSOCIATED VIRUS COMPOSITIONS AND METHODS OF USE THEREOF

(71) Applicant: Homology Medicines, Inc., Bedford, MA (US)

(72) Inventor: Serena Nicole DOLLIVE, Bedford, MA (US)

(21) Appl. No.: 17/806,409

(22) Filed: Jun. 10, 2022

Related U.S. Application Data
(63) Continuation of application No. PCT/US2020/064214, filed on Dec. 10, 2020.
(60) Provisional application No. 62/946,164, filed on Dec. 10, 2019.

(51) Int. Cl. C07K 14/005

(52) U.S. Cl. CPC C07K 14/005; C12N 2750/14122; C12N 2750/14143

(57) ABSTRACT

Provided herein are novel adeno-associated virus (AAV) capsids, compositions (e.g., rAAV) comprising the capsids, and nucleic acids encoding the capsids. Also provided are methods of making and using the capsids and compositions disclosed herein. The rAAV disclosed herein are particularly useful for gene transfer applications where high transduction efficiency is required (e.g., gene therapy).

### CROSS-REFERENCE TO RELATED APPLICATIONS

This application is a continuation of International Application No. PCT/US2020/064214, filed Dec. 10, 2020, which claims the benefit of U.S. Provisional Application No. 62/946,164, filed Dec. 10, 2019, the disclosure of each of which is hereby incorporated by reference in its entirety.

### SEQUENCE LISTING

This application contains a sequence listing which has been submitted electronically in ASCII format and is hereby incorporated by reference in its entirety (said ASCII copy, created on Nov. 30, 2020, is named “HMW-032US_ST25.txt” and is 30,384 bytes in size).

### BACKGROUND

Adeno-associated virus (AAV) possesses unique features that make it attractive as a vector for delivering foreign DNA into cells for the purposes of gene therapy. For example: AAV infection of cells in culture is non-cytopathic, and natural infection of humans and other animals is silent; AAV infects many different mammalian tissues in vivo; the AAV proviral genome is infectious as cloned DNA in plasmids, which makes construction of recombinant AAV genomes feasible; and, because the signals directing AAV replication, genome encapsidation and integration are contained within the inverted terminal repeats (ITRs) of the AAV genome, essentially all of the internal 4.3 kb of the AVV genome (encoding the replication and structural capsid proteins, rep-cap) can be replaced with heterologous nucleic acid sequences, such as a transgene expression cassette.

There is a need in the art for novel AAVs that exhibit a high transduction efficiency in a variety of clinically relevant cell types, for use in gene therapy applications.

### SUMMARY

Provided herein are novel adeno-associated virus (AAV) capsids, compositions (e.g., rAAV) comprising the capsids, and nucleic acids encoding the capsids. Also provided are methods of making and using the capsids and compositions disclosed herein. The AAV capsids provided herein mediate high transduction efficiency in a variety of clinically relevant cell types, including a variety of brain cell types. Moreover, rAAV comprising these novel capsids can cross the blood-brain barrier after systemic delivery, and transduce a variety of cell types in the brain. It is also believed that these novel AAV capsid proteins will be well tolerated when administered to human subjects. Accordingly, the rAAV disclosed herein are particularly useful for gene therapy applications.

Accordingly, in one aspect, the instant disclosure provides an AAV capsid protein comprising an amino acid sequence having at least 95% sequence identity with the amino acid sequence of amino acids 203-736 of SEQ ID NO:1, wherein the amino acid in the capsid protein corresponding to amino acid 412 of SEQ ID NO:1 is T.

In certain embodiments, the capsid protein comprises an amino acid sequence having at least 99% sequence identity with the amino acid sequence of amino acids 203-736 of SEQ ID NO:1. In certain embodiments, the capsid protein comprises the amino acid sequence of amino acids 203-736 of SEQ ID NO:1. In certain embodiments, the amino acid sequence of the capsid protein consists of the amino acid sequence of amino acids 203-736 of SEQ ID NO:1.

In another aspect, the instant disclosure provides an AAV capsid protein comprising an amino acid sequence having at least 95% sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; or the amino acid in the capsid protein corresponding to amino acid 412 of SEQ ID NO:1 is T.

In certain embodiments, the capsid protein comprises an amino acid sequence having at least 99% sequence identity with the amino acid sequence of amino acids 138-736 of SEQ ID NO:1. In certain embodiments, the capsid protein comprises the amino acid sequence of amino acids 138-736 of SEQ ID NO:1. In certain embodiments, the amino acid sequence of the capsid protein consists of the amino acid sequence of amino acids 138-736 of SEQ ID NO:1.

In another aspect, the instant disclosure provides an AAV capsid protein comprising an amino acid sequence having at least 95% sequence identity with the amino acid sequence of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; or the amino acid in the capsid protein corresponding to amino acid 412 of SEQ ID NO:1 is T.

In certain embodiments, the capsid protein comprises an amino acid sequence having at least 99% sequence identity with the amino acid sequence of SEQ ID NO:1. In certain embodiments, the capsid protein comprises the amino acid sequence of SEQ ID NO:1. In certain embodiments, the amino acid sequence of the capsid protein consists of the amino acid sequence of SEQ ID NO:1.

In another aspect, the instant disclosure provides an isolated polynucleotide encoding a capsid protein as described herein.

In another aspect, the instant disclosure provides a vector comprising a polynucleotide as described herein.

In certain embodiments, the vector is a plasmid or a viral vector. In certain embodiments, the viral vector is a retrovirus vector, a herpes virus vector, a baculovirus vector, or an adenovirus vector). In certain embodiments, the vector is an expression vector.

In another aspect, the instant disclosure provides a recombinant cell comprising a polynucleotide as described herein, or a vector as described herein.

In another aspect, the instant disclosure provides a method of producing an AAV capsid protein, the method comprising culturing a recombinant cell described herein under conditions whereby a polynucleotide as described herein is expressed and a capsid as described herein is produced.

In another aspect, the instant disclosure provides a recombinant adeno-associated virus (rAAV) comprising: a capsid comprising one or more of the capsid proteins as described herein; and an rAAV genome.

In certain embodiments, the rAAV genome comprises a transgene. In certain embodiments, the transgene encodes a polypeptide. In certain embodiments, the transgene encodes an miRNA, shRNA, siRNA, antisense RNA, gRNA, antagomir, miRNA sponge, RNA aptazyme, RNA aptamer, lncRNA, ribozyme or mRNA. In certain embodiments, the transgene is operably linked to a transcriptional regulatory element. In certain embodiments, the rAAV genome comprises an editing genome.

In another aspect, the instant disclosure provides a method for transducing a cell, the method comprising contacting the cell with an rAAV as described herein under conditions whereby the cell is transduced.

In another aspect, the instant disclosure provides a method for expressing a transgene in a cell, the method comprising contacting the cell with an rAAV as described herein under conditions whereby the cell is transduced and the transgene is expressed.

In another aspect, the instant disclosure provides a method for editing a target locus in a genome of a cell, the method comprising contacting an rAAV as described herein under conditions whereby the cell is transduced and the target locus is edited.

In certain embodiments, the cell is a blood, liver, heart, joint tissue, muscle, brain, kidney, or lung cell. In certain embodiments, the cell is a cell of the central nervous system or a cell of the peripheral nervous system.

In certain embodiments, the method is performed ex-vivo or in vitro. In certain embodiments, the cell is in a subject and the rAAV is administered to the subject. In certain embodiments, the rAAV is administered to the subject intravenously, intraperitoneally, subcutaneously, intramuscularly, intrathecally, or intradermally. In certain embodiments, the subject is a human subject.

In another aspect, the instant disclosure provides a packaging system for preparation of an rAAV, wherein the packaging system comprises: (a) a first nucleotide sequence encoding one or more AAV Rep proteins; (b) a second nucleotide sequence encoding a capsid protein as described herein; and (c) a third nucleotide sequence comprising an rAAV genome sequence.

In certain embodiments, the packaging system comprises a first vector comprising the first nucleotide sequence and the second nucleotide sequence, and a second vector comprising the third nucleotide sequence. In certain embodiments, the packaging system further comprises a forth nucleotide sequence comprising one or more helper virus genes. In certain embodiments, the forth nucleotide sequence is comprised within a third vector. In certain embodiments, the forth nucleotide sequence comprises one or more genes from a virus selected from the group consisting of adenovirus, herpesvirus, vaccinia virus, and cytomegalovirus (CMV). In certain embodiments, the first vector, second vector, and/or the third vector is a plasmid.

In another aspect, the instant disclosure provides a method for recombinant preparation of an rAAV, the method comprising introducing a packaging system as described herein into a cell under conditions whereby the rAAV is produced.

In another aspect, the instant disclosure provides an rAAV as described herein for use in medicine, for use as therapy, or for use as a medicament.

### BRIEF DESCRIPTION OF THE DRAWINGS

FIGS. 1A, 1B, and 1C are graphs showing Capsid X productivity in crude lysate according to the experimental designs set forth in Table 4. FIG. 1A shows Capsid X titer as determined by ddPCR. FIG. 1B shows Capsid X titer as determined by ELISA. FIG. 1C shows percentage of full capsids detected, as determined using ELISA and ddPCR values.

FIGS. 2A, 2B, and 2C are graphs showing Capsid X productivity in batch purified products according to the experimental designs set forth in Table 4. FIG. 2A shows Capsid X titer as determined by ddPCR. FIG. 2B shows Capsid X titer as determined by ELISA. FIG. 2C shows percentage of full capsids detected, as determined using ELISA and ddPCR values.

### DETAILED DESCRIPTION

The instant disclosure provides novel adeno-associated virus (AAV) capsids, compositions (e.g., rAAV) comprising the capsids, and nucleic acids encoding the capsids. Also provided are methods of making and using the capsids and compositions disclosed herein. The rAAV disclosed herein are particularly useful for gene transfer applications where high transduction efficiency is required (e.g., gene therapy).

### I. DEFINITIONS

As used herein, the term “AAV” is a standard abbreviation for adeno-associated virus.

As used herein, the term “recombinant adeno-associated virus” or “rAAV” refers to an AAV comprising a genome lacking functional rep and cap genes.

As used herein, the term “cap gene” refers to a nucleic acid sequence that encodes a capsid protein

As used herein, the term “rep gene” refers to the nucleic acid sequences that encode the non-structural proteins (e.g., rep78, rep68, rep52 and rep40) required for the replication and production of an AAV.

As used herein, the term “rAAV genome” refers to a nucleic acid molecule (e.g., DNA and/or RNA) comprising the genome sequence of an rAAV. In certain embodiments, an rAAV genome comprises or consists of a single stranded DNA molecule. In certain embodiments, an rAAV genome comprises or consists of a double stranded DNA molecule (e.g., a self-complementary rAAV genome).

As used herein, an “isolated” polynucleotide is one which is separated from other nucleic acid molecules which are present in the natural source of the polynucleotide (e.g., a wild type AAV genome).

As used herein, the term “editing genome” refers to an rAAV genome, comprising an editing element for editing a target locus, flanked by (i) a 5′ homology arm sequence 5′ of the editing element having homology to a first genomic region 5′ to the target locus; and (ii) a 3′ homology arm nucleotide 3′ of the editing element having homology to a second genomic region 3′ to the target locus. An editing genome is capable of integrating an editing element via homologous recombination into a target locus (e.g., a human target locus) to edit that locus (e.g., to correct a genetic defect in a gene). The skilled artisan will appreciate that the portion of an editing genome comprising a 5′ homology arm, editing element, a 3′ homology arm can be in the sense or anti sense orientation relative to the target locus.

As used herein, the term “editing element” refers to the portion of an editing genome that when integrated by homologous recombination at a target locus modifies the target locus. An editing element can mediate insertion, deletion, or substitution of one or more nucleotides at the target locus.

As used herein, the term “target locus” refers to a region of a chromosome or an internucleotide bond (e.g., a region or an internucleotide bond of a human gene) that is modified by an editing element.

As used herein, the term “homology arm” refers to a portion of an editing genome positioned 5′ or 3′ of an editing element that is substantially identical (e.g., 100% identical) to a portion of the genome sequence flanking a target locus. In certain embodiments, the target locus is in a human gene, and the homology arm comprises a sequence substantially identical to the genome sequence flanking the target locus in the human gene. In certain embodiments, the target locus in an intergenic region of a genome (e.g., human genome).

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, a “vector” refers to a nucleic acid molecule that is a vehicle for introducing a nucleic acid molecule (e.g., a polynucleotide disclosed herein) into a cell.

As used herein, an “expression vector” refers to a vector comprising transcriptional regulatory elements operably linked to a gene of interest (e.g., a polynucleotide disclosed herein) that facilitate the expression of the gene of interest in a cell and/or a cell free expression system.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “transgene” refers to a non-AAV nucleic acid sequence that encodes a polypeptide or non-coding RNA (e.g., an miRNA, shRNA, siRNA, antisense RNA, gRNA, antagomir, miRNA sponge, RNA aptazyme, or RNA aptamer).

As used herein, the term “operably linked” is used to describe the connection between a TRE and a polynucleotide sequence (e.g., a transgene disclosed herein) to be transcribed. Typically, gene expression is placed under the control of a TRE comprising one or more promoter and/or enhancer elements. The transgene is “operably linked” to the TRE if the transcription of the transgene is controlled or influenced by the TRE. The promoter and enhancer elements of the TRE may be in any orientation and/or distance from the transgene, as long as the desired transcriptional activity is obtained. In certain embodiments, the TRE is upstream from the transgene.

As used herein, the term “effective amount” in the context of the administration of an AAV to a subject refers to the amount of the AAV that achieves a desired prophylactic or therapeutic effect.

### II. AAV CAPSID PROTEINS

In one aspect, the instant disclosure provides a polypeptide (e.g., an AAV capsid protein) comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 203-736 of SEQ ID NO:1, wherein the amino acid in the capsid protein corresponding to amino acid 412 of SEQ ID NO:1 is T. In certain embodiments, the polypeptide (e.g., AAV capsid protein) comprises the amino acid sequence of amino acids 203-736 of SEQ ID NO:1. In certain embodiments, the amino acid sequence of the polypeptide (e.g., AAV capsid protein) consists of the amino acid sequence of amino acids 203-736 of SEQ ID NO:1.

In certain embodiments, the instant disclosure provides a polypeptide (e.g., an AAV capsid protein) comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T. In certain embodiments, the polypeptide (e.g., AAV capsid protein) comprises the amino acid sequence of amino acids 138-736 of SEQ ID NO:1. In certain embodiments, the amino acid sequence of the polypeptide (e.g., AAV capsid protein) consists of the amino acid sequence of amino acids 138-736 of SEQ ID NO:1.

In certain embodiments, the instant disclosure provides a polypeptide (e.g., an AAV capsid protein) comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 1-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T. In certain embodiments, the polypeptide (e.g., AAV capsid protein) comprises the amino acid sequence of amino acids 1-736 of SEQ ID NO:1. In certain embodiments, the amino acid sequence of the polypeptide (e.g., AAV capsid protein) consists of the amino acid sequence of amino acids 1-736 of SEQ ID NO:1.

### III. POLYNUCLEOTIDES, VECTORS, AND METHODS OF PRODUCING AAV CAPSIDS

In another aspect, the instant disclosure provides polynucleotides (e.g., an isolated polynucleotides) encoding a polypeptide (e.g., an AAV capsid protein) disclosed herein.

In certain embodiments, the instant disclosure provides a polynucleotide encoding a polypeptide (e.g., an AAV capsid protein) comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 203-736 of SEQ ID NO:1, wherein the amino acid in the capsid protein corresponding to amino acid 412 of SEQ ID NO:1 is T. In certain embodiments, the polypeptide (e.g., AAV capsid protein) comprises the amino acid sequence of amino acids 203-736 of SEQ ID NO:1. In certain embodiments, the amino acid sequence of the polypeptide (e.g., AAV capsid protein) consists of the amino acid sequence of amino acids 203-736 of SEQ ID NO:1.

In certain embodiments, the instant disclosure provides a polynucleotide encoding a polypeptide (e.g., an AAV capsid protein) comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T. In certain embodiments, the polypeptide (e.g., AAV capsid protein) comprises the amino acid sequence of amino acids 138-736 of SEQ ID NO:1. In certain embodiments, the amino acid sequence of the polypeptide (e.g., AAV capsid protein) consists of the amino acid sequence of amino acids 138-736 of SEQ ID NO:1.

In certain embodiments, the instant disclosure provides a polynucleotide encoding a polypeptide (e.g., an AAV capsid protein) comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 1-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T. In certain embodiments, the polypeptide (e.g., AAV capsid protein) comprises the amino acid sequence of amino acids 1-736 of SEQ ID NO:1. In certain embodiments, the amino acid sequence of the polypeptide (e.g., AAV capsid protein) consists of the amino acid sequence of amino acids 1-736 of SEQ ID NO:1.

In certain embodiments, the polynucleotide is optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of the encoded capsid protein relative to the expression of the capsid encoded by polynucleotides that have not been optimized.

In another aspect the instant disclosure provides a vector comprising a polynucleotide disclosed herein. Suitable vectors, include, without limitation, plasmids, viruses, cosmids, artificial chromosomes, linear DNA, and mRNA. In certain embodiments, the vector is a plasmid or a viral vector. In certain embodiments, the vector is a retrovirus vector, a herpes virus vector, a baculovirus vector, or an adenovirus vector. In certain embodiments, the vector is an expression vector.

Vectors (e.g., expression vectors) can be introduced into cells (using any techniques known in the art) for propagation of the vector and/or for expression of an AAV capsid protein encoded by the vector. Accordingly, in another aspect, the instant disclosure provides a recombinant cell comprising a polynucleotide or a vector (e.g., an expression vector) disclosed herein. And further, in another aspect, the instant disclosure provides a method of producing an AAV capsid protein, the method comprising culturing the recombinant cell under conditions whereby the polynucleotide is expressed and the capsid is produced.

A variety of host cells and expression vector systems can be utilized to express the capsid proteins described herein. Such expression systems represent vehicles by which the coding sequences of interest can be produced and subsequently purified, but also represent cells which can, when transformed or transfected with the appropriate nucleotide coding sequences, express a capsid protein described herein in situ. These include but are not limited to microorganisms such as bacteria (e.g., E. coli and B. subtilis) transformed with, e.g., recombinant bacteriophage DNA, plasmid DNA or cosmid DNA expression vectors containing capsid protein coding sequences; yeast (e.g., Saccharomyces Pichia) transformed with, e.g., recombinant yeast expression vectors containing capsid protein coding sequences; insect cell systems infected with, e.g., recombinant virus expression vectors (e.g., baculovirus) containing capsid protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing capsid protein coding sequences; or mammalian cell systems (e.g., COS (e.g., COS1 or COS), CHO, BHK, MDCK, HEK 293, NSO, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, cells for expressing the capsid proteins described herein are human cells, e.g., human cell lines. In certain embodiments, a mammalian expression vector is pOptiVEC™ or pcDNA3.3. In certain embodiments, bacterial cells such as Escherichia coli, or eukaryotic cells (e.g., mammalian cells), are used for the expression of a capsid protein. For example, mammalian cells such as CHO or HEK293 cells, in conjunction with a vector such as the major intermediate early gene promoter element from human cytomegalovirus is an effective expression system for capsid proteins disclosed herein.

In bacterial systems, a number of expression vectors can be advantageously selected depending upon the use intended for the capsid protein being expressed. For example, when a large quantity of a capsid protein is to be produced, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the capsid protein coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The capsid protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the capsid protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the capsid protein molecule in infected hosts (e.g., see Logan J & Shenk T (1984) PNAS 81(12): 3655-9, which is herein incorporated by reference in its entirety). Specific initiation signals can also be required for efficient translation of inserted capsid protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NSO (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

For long-term, high-yield production of recombinant proteins, stable expression cells can be generated. For example, cell lines which stably express a capsid protein described herein can be engineered.

In certain embodiments, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a capsid protein described herein or a fragment thereof.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, N Y (1993); Kriegler M, Gene Transfer and Expression, A Laboratory Manual, Stockton Press, N Y (1990); and in Chapters 12 and 13, Dracopoli N C et al., (eds.), Current Protocols in Human Genetics, John Wiley & Sons, N Y (1994); Colbère-Garapin F et al., (1981) J Mol Biol 150: 1-14, all of which are herein incorporated by reference in their entireties.

### IV. AAV COMPOSITIONS

In another aspect, the instant disclosure provides an rAAV comprising a capsid comprising one of more of the novel capsid proteins disclosed herein.

In certain embodiments, the rAAV comprises a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 203-736 of SEQ ID NO:1, wherein the amino acid in the capsid protein corresponding to amino acid 412 of SEQ ID NO:1 is T. In certain embodiments, AAV capsid protein comprises the amino acid sequence of amino acids 203-736 of SEQ ID NO:1. In certain embodiments, the amino acid sequence of the AAV capsid protein consists of the amino acid sequence of amino acids 203-736 of SEQ ID NO:1.

In certain embodiments, the rAAV comprises a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T. In certain embodiments, the polypeptide (e.g., AAV capsid protein) comprises the amino acid sequence of amino acids 138-736 of SEQ ID NO:1. In certain embodiments, the amino acid sequence of the polypeptide (e.g., AAV capsid protein) consists of the amino acid sequence of amino acids 138-736 of SEQ ID NO:1.

In certain embodiments, the rAAV comprises a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 1-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T. In certain embodiments, the polypeptide (e.g., AAV capsid protein) comprises the amino acid sequence of amino acids 1-736 of SEQ ID NO:1. In certain embodiments, the amino acid sequence of the polypeptide (e.g., AAV capsid protein) consists of the amino acid sequence of amino acids 1-736 of SEQ ID NO:1.

In certain embodiments, the rAAV comprises two or more of:

(a) a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 203-736 of SEQ ID NO:1, wherein the amino acid in the capsid protein corresponding to amino acid 412 of SEQ ID NO:1 is T, optionally wherein AAV capsid protein comprises the amino acid sequence of amino acids 203-736 of SEQ ID NO:1, optionally wherein the amino acid sequence of the AAV capsid protein consists of the amino acid sequence of amino acids 203-736 of SEQ ID NO:1;
(b) a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T, optionally wherein AAV capsid protein comprises the amino acid sequence of amino acids 138-736 of SEQ ID NO:1, optionally wherein the amino acid sequence of the AAV capsid protein consists of the amino acid sequence of amino acids 138-736 of SEQ ID NO:1, or
(c) a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 1-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T, optionally wherein AAV capsid protein comprises the amino acid sequence of amino acids 138-736 of SEQ ID NO:1, optionally wherein the amino acid sequence of the AAV capsid protein consists of the amino acid sequence of amino acids 138-736 of SEQ ID NO:1.

In certain embodiments, the rAAV comprises:

(a) a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 203-736 of SEQ ID NO:1, wherein the amino acid in the capsid protein corresponding to amino acid 412 of SEQ ID NO:1 is T, optionally wherein AAV capsid protein comprises the amino acid sequence of amino acids 203-736 of SEQ ID NO:1, optionally wherein the amino acid sequence of the AAV capsid protein consists of the amino acid sequence of amino acids 203-736 of SEQ ID NO:1;
(b) a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T, optionally wherein AAV capsid protein comprises the amino acid sequence of amino acids 138-736 of SEQ ID NO:1, optionally wherein the amino acid sequence of the AAV capsid protein consists of the amino acid sequence of amino acids 138-736 of SEQ ID NO:1, and
(c) a capsid comprising an AAV capsid protein comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the amino acid sequence of amino acids 1-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; and/or the amino acid in the capsid protein corresponding to amino acid 412 of SEQ ID NO:1 is T. In certain embodiments, the amino acid in the capsid protein corresponding to amino acid 146 of SEQ ID NO:1 is I; the amino acid in the capsid protein corresponding to amino acid 157 of SEQ ID NO:1 is V; and the amino acid in the capsid protein corresponding to amino acid 412 of SEQ ID NO:1 is T, optionally wherein AAV capsid protein comprises the amino acid sequence of amino acids 138-736 of SEQ ID NO:1, optionally wherein the amino acid sequence of the AAV capsid protein consists of the amino acid sequence of amino acids 138-736 of SEQ ID NO:1.

The rAAVs disclosed herein generally comprise a recombinant genome (e.g., an rAAV genome) packaged within the capsid. The rAAV genome can be of any type that is capable of being packages within an AAV capsid disclosed herein. For example, in certain embodiments, the rAAV genome is a single-stranded DNA genome. In certain embodiments, the rAAV genome is a self-complementary genome, for example as described in U.S. Pat. No. 7,790,154, which is hereby incorporated by reference in its entirety

In certain embodiments, the rAAV genome comprises a transgene. In certain embodiments the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-α receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groa/IL-8, RANTES, MIP-1a, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes a nuclease. Suitable nucleases include, without limitation, zinc fingers nucleases (ZFN) (see e.g., Porteus, and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see e.g., Wood et al. (2011) Science 333:307; Boch et al. (2009) Science 326:1509-1512; Moscou and Bogdanove (2009) Science 326; 1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C(Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV genome comprises a TRE operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441), CMV early enhancer/chicken β-actin (CBA) promoter/rabbit β-globin intron (CAG) (Miyazaki et al. (1989) Gene 79(2): 269-277, CB^{SB} (Jacobson et al. (2006) Molecular Therapy 13(6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32(3): 409-417, mitochondrial heavy-strand promoter (Loderio et al. (2012) PNAS 109(17): 6513-6518), ubiquitin promoter (Wulff et al. (1990) FEBS Letters 261: 101-105). In certain embodiments, the TRE comprises a cytomegalovirus (CMV) promoter/enhancer (e.g., comprising a nucleotide sequence at least 90%, 91%, 92%, 93%, 94%, 95%, 96%, 97%, 98%, 99%, or 100% identical to SEQ ID NO:2 or 3), an SV40 promoter, a chicken beta actin (CBA) promoter (e.g., comprising a nucleotide sequence at least 90%, 91%, 92%, 93%, 94%, 95%, 96%, 97%, 98%, 99%, or 100% identical to SEQ ID NO:4 or 5), a smCBA promoter (e.g., comprising a nucleotide sequence at least 90%, 91%, 92%, 93%, 94%, 95%, 96%, 97%, 98%, 99%, or 100% identical to SEQ ID NO:6), a human elongation factor 1 alpha (EF1α) promoter (e.g., comprising a nucleotide sequence at least 90%, 91%, 92%, 93%, 94%, 95%, 96%, 97%, 98%, 99%, or 100% identical to SEQ ID NO:7), a minute virus of mouse (MVM) intron which comprises transcription factor binding sites (e.g., comprising a nucleotide sequence at least 90%, 91%, 92%, 93%, 94%, 95%, 96%, 97%, 98%, 99%, or 100% identical to SEQ ID NO:8 or 9), a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element (e.g., comprising a nucleotide sequence at least 90%, 91%, 92%, 93%, 94%, 95%, 96%, 97%, 98%, 99%, or 100% identical to SEQ ID NO:10 or 11), a human calmodulin 1 (CALM1) promoter (e.g., comprising a nucleotide sequence at least 90%, 91%, 92%, 93%, 94%, 95%, 96%, 97%, 98%, 99%, or 100% identical to SEQ ID NO:12), a human ApoE/C-I hepatic control region (HCR1) (e.g., comprising a nucleotide sequence at least 90%, 91%, 92%, 93%, 94%, 95%, 96%, 97%, 98%, 99%, or 100% identical to SEQ ID NO:13), a human α1-antitrypsin (hAAT) promoter (e.g., comprising a nucleotide sequence at least 90%, 91%, 92%, 93%, 94%, 95%, 96%, 97%, 98%, 99%, or 100% identical to SEQ ID NO:14, 15, or 16), an extended HCR1 (e.g., comprising a nucleotide sequence at least 90%, 91%, 92%, 93%, 94%, 95%, 96%, 97%, 98%, 99%, or 100% identical to SEQ ID NO:22), a HS-CRM8 element of an hAAT promoter (e.g., comprising a nucleotide sequence at least 90%, 91%, 92%, 93%, 94%, 95%, 96%, 97%, 98%, 99%, or 100% identical to SEQ ID NO:23), a human transthyretin (TTR) promoter (e.g., comprising a nucleotide sequence at least 90%, 91%, 92%, 93%, 94%, 95%, 96%, 97%, 98%, 99%, or 100% identical to SEQ ID NO:17), and/or a human Methyl-CpG Binding Protein 2 (MeCP2) promoter. Any of the TREs described herein can be combined in any order to drive efficient transcription. For example, a transfer genome may comprise a TRE comprising a CMV enhancer, a CBA promoter, and the splice acceptor from exon 3 of the rabbit beta-globin gene, collectively called a CAG promoter (e.g., comprising a nucleotide sequence at least 90%, 91%, 92%, 93%, 94%, 95%, 96%, 97%, 98%, 99%, or 100% identical to SEQ ID NO:18). For example, a transfer genome may comprise a TRE comprising a hybrid of CMV enhancer and CBA promoter followed by a splice donor and splice acceptor, collectively called a CASI promoter region (e.g., comprising a nucleotide sequence at least 90%, 91%, 92%, 93%, 94%, 95%, 96%, 97%, 98%, 99%, or 100% identical to SEQ ID NO:19). For example, a transfer genome may comprise a TRE comprising a HCR1 and hAAT promoter (also referred to as an LP1 promoter, e.g., comprising a nucleotide sequence at least 90%, 91%, 92%, 93%, 94%, 95%, 96%, 97%, 98%, 99%, or 100% identical to SEQ ID NO:20).

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the rAAV genome comprises an editing genome. Editing genomes can be used to edit the genome of a cell by homologous recombination of the editing genome with a genomic region surrounding a target locus in the cell. In certain embodiments, the editing genome is designed to correct a genetic defect in a gene by homologous recombination. Suitable target genes for editing using an editing genome include, without limitation, phenylalanine hydroxylase (PAH), cystic fibrosis conductance transmembrane regulator (CFTR), beta hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y).

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

### V. METHODS OF USE

In another aspect, the instant disclosure provides methods for transducing a cell. The methods generally comprise contacting the cell with a rAAV disclosed herein under conditions whereby the cell is transduced.

The rAAV disclosed herein can comprise a transgene under the control of a TRE. Accordingly, in certain embodiments, the instant disclosure provides methods for expressing a transgene in a cell, the method generally comprising contacting the cell with such an rAAV under conditions whereby the cell is transduced and the transgene is expressed. The transgene can encode a polypeptide and/or an RNA molecule, as described herein. Accordingly, in certain embodiments, the instant disclosure provides methods for producing a polypeptide and/or an RNA molecule in a cell, the method generally comprising contacting the cell with such an rAAV under conditions whereby the cell is transduced and the polypeptide and/or an RNA molecule is produced.

The rAAV disclosed herein can comprise an editing genome. rAAV comprising editing genomes can be used to edit the genome of a cell by homologous recombination of the editing genome with a homologous target locus in the cell. Accordingly, in certain embodiments, the instant disclosure provides a method for editing a target locus in a genome of a cell, the method generally comprising contacting the cell with such an rAAV under conditions whereby the cell is transduced and the target locus is edited.

The rAAV disclosed herein can be used to transduce cells in vitro, in vivo and ex vivo. Cells suitable for being transduced by the rAAV disclosed herein include, without limitation, blood, liver, heart, joint tissue, muscle, brain, kidney, or lung cells. In certain embodiments, the cell is a cell of the central nervous system or peripheral nervous system.

The rAAV disclosed herein can be administered to a subject (e.g., a human subject) by all routes suitable for an rAAV, including, without limitation, intravenously, intraperitoneally, subcutaneously, intramuscularly, intrathecally, or intradermally.

In another aspect, the invention provides an rAAV as disclosed herein for use in medicine. In another aspect, the invention provides an rAAV as disclosed herein for use as therapy. In another aspect, the invention provides an rAAV as disclosed herein for use as a medicament.

### VI. ADENO-ASSOCIATED VIRUS PACKAGING SYSTEMS

In another aspect, the instant disclosure provides packaging systems for recombinant preparation of a recombinant adeno-associated virus (rAAV) disclosed herein. Such packaging systems generally comprise: first nucleotide encoding one or more AAV Rep proteins; a second nucleotide encoding a capsid protein of any of the AAVs as disclosed herein; and a third nucleotide sequence comprising any of the rAAV genome sequences as disclosed herein, wherein the packaging system is operative in a cell for enclosing the transfer genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In certain embodiments of the packaging system, the Rep nucleotide sequence encodes an AAV2 Rep protein. Suitable AAV2 Rep proteins may include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO:21, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO:21.

In certain embodiments of the packaging system, the packaging system further comprises a forth nucleotide sequence comprising one or more helper virus genes. In certain embodiments, the forth nucleotide sequence comprises adenoviral E2, E4 and VA genes. In certain embodiments of the packaging system, the packaging system further comprises a third vector (e.g., a helper virus vector), comprising the forth nucleotide sequence. The third vector may be an independent third vector, integral with the first vector, or integral with the second vector.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the adenovirus genome comprises one or more adenovirus RNA genes selected from the group consisting of E2, E4 and VA. In certain embodiments of the packaging system, where the helper virus is HSV, the HSV genome comprises one or more of HSV genes selected from the group consisting of UL5/8/52, ICPO, ICP4, ICP22 and UL30/UL42.

In certain embodiments of the packaging system, the first, second, and/or third vector are contained within one or more plasmids. In certain embodiments, the first vector and the third vector are contained within a first plasmid. In certain embodiments the second vector and the third vector are contained within a second plasmid.

In certain embodiments of the packaging system, the first, second, and/or third vector are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g. with one or more recombinant helper viruses, such as a adenovirus, poxvirus (such as vaccinia virus), herpes virus (including HSV, cytomegalovirus, or baculovirus, containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more capsid proteins as described herein, and with a transfer genome as described herein being delivered in the form of a plasmid or a recombinant helper virus).

Accordingly, the instant disclosure provides a packaging system for preparation of a rAAV, wherein the packaging system comprises: a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a forth nucleotide sequence comprising one or more helper virus genes (e.g., adenoviral E2, E4 and VA genes).

### VI. EXAMPLES

The following examples are offered by way of illustration, and not by way of limitation.

### Example 1: Identification of a Novel AAV Capsid Protein

A novel AAV capsid protein sequence (Capsid X) was discovered by in silico sequence database analysis. Specifically, a consensus sequence of the hematopoietic stem cell-derived AAVHSC capsid sequences (as described in U.S. Pat. No. 9,890,396, which is hereby incorporated by reference in its entirety) was generated. This consensus sequence was searched against various sequence databases available through the National Center for Biotechnology Information (NCBI) BLAST database, and a source contig was generated from the search results. This contig was aligned with Clade F capsid sequences, including all 15 AAVHSCs, AAV9, hu.31, and hu.32. The novel capsid sequence was found to be phylogenetically closer to the AAVHSC capsids than to other Clade F capsids.

### Example 2: AAV Packaging Using Capsid X

The ability of Capsid X to package into a functional AAV was tested. Specifically, HEK293 were seeded at a density of 2×10⁶ cells/mL in 100 mL of culture, and placed into a shaking incubator for 1 hour at 37° C. and 125 rpm. Cell were then transfected in the shake flasks with the following 3 plasmids using polyethylenimine (PEI): scGFP (a plasmid encoding a self-complementary AAV genome comprising AAV2 ITRs and a promoter operably linked to enhanced green fluorescent protein (EGFP), as described in U.S. Pat. No. 8,628,966, which is incorporated by reference herein in its entirety) used as the AAV genome, the pHelper plasmid (Agilent) which provides the necessary Adenovirus helper functions for rAAV production, and a plasmid encoding the rep (AAV2) and cap (either AAVHSC15 or Capsid X) genes. 72 hours after transfection, 5 mL of each shake flask was added to a 15 mL conical tube and cells pelleted at 2000 RPM for 20 minutes. 1 mL of supernatant was used to measure cell metabolites. The remaining supernatant was then discarded, and the pellet was resuspended in 5 mL of lysis buffer (containing Tris-NaCl and Triton) diluted 1:10 with MgCl₂/Benzonase added, and incubated for 1 hour at 37° C. After incubation, the crude cell lysate was clarified at 4300×g for 20 minutes and samples of the clarified lysate were used for ddPCR and Capsid ELISA, to determine viral genome titer and capsid titer, respectively.

In order to investigate whether Capsid X was properly packaged, Western blot analysis of the clarified lysate was also performed, with antibodies A1, B2, and VP51 being used to detect capsid protein. Antibodies A1, B2, and VP51 are broadly reactive with known AAVs. The A1 antibody reacts with a conserved region in the N-terminal region while the B1 antibody reacts with a conserved region in the C-terminal region of AAVs. The VP51 antibody broadly reacts with most of known AAVs. In this experiment, VP1, VP2, and VP3 bands were each detected for Capsid X, and these bands were comparable to those detected for the AAVHSC15 control.

Table 1 shows the average ddPCR titer, ELISA titer, and packaging capacity (% full) determined for Capsid X and the AAVHSC15 control. ELISA was performed using the ADK9 antibody, which is specific to clade F capsids.

TABLE 1
Titer and Packaging Efficiency of Capsid X

| | Average ddPCR Titer | ELISA Titer | |
|---|---|---|---|
| rAAV | (vg/mL)* | (capsids/mL)* | % Full |
| Control (AAVHSC15) | 4.08 × 10¹⁰ | 3.38 × 10¹¹ | 12.1% |
| Capsid X | 3.38 × 10¹⁰ | 2.66 × 10¹¹ | 12.7% |

*viral genomes or capsids per mL in HEK293 cells suspension culture

### Example 3: Transduction of Huh7 and HeLa Cells

Huh7 (Creative Bio) and HeLa (Sigma) cells were each cultured in DMEM (Gibco) supplemented with 10% fetal bovine serum (FBS; Gibco) and penicillin-streptomycin (Gibco), and plated onto 48-well plates. When the cells reached approximately 90% confluency, the cells were transduced with Capsid X, AAVHSC15, or AAV2 rAAV, each carrying the scGFP genome described above. Cells were transduced at a multiplicity of infection (MOI) of 150,000 by addition of virus to the cell culture media. Wells were imaged and analyzed for GFP expression at 1, 2, and 6 days following transduction. GFP expression in cells that were transduced with the Capsid X virus was found to be similar to that observed in cells transduced with the AAVHSC15 virus.

### Example 4: Characterization of Capsid X in the Murine Nervous System

Mouse central nervous system (CNS) tropism for Capsid X was characterized. Wild type mice were administered intravenously, via tail vein, a self-complementary AAV genome comprising an eGFP expression cassette packaged in Capsid X (CapsidX-eGFP), or an eGFP expression cassette packaged in AAVHSC15 (HSC15-eGFP). Analysis was performed on one section of brain per animal, with six animals per analyzed group. CNS cell types were identified by histological profile.

A uniform rostro-caudal distribution of eGFP signal was detected, and no differences were observed between male and female animals administered either CapsidX-eGFP or HSC15-eGFP. eGFP expression was detected in glial and neuronal cells of the murine CNS indicating that these cells were transduced by CapsidX-eGFP. eGFP+ vasculature was found to be more abundant in brain sections of CapsidX-eGFP administered mice.

Various regions of the CNS were tested for eGFP expression. A summary of findings is provided in Table 2 below, with “+” indicating detection of eGFP.

TABLE 2
Capsid X Tropism Characterization in the Murine CNS

| | Capsid X | | | | AAVHSC15 | | | |
|---|---|---|---|---|---|---|---|---|
| | Glia | | | | Glia | | | |
| | | Oligo- | | | | Oligo- | | |
| Region | Astrocytes | dendrocytes | Microglia | Neurons | Astrocytes | dendrocytes | Microglia | Neurons |
| Olfactory bulbs | + | | + | | + | | + | |
| Secondary | + | | + | | + | | + | |
| motor cortex | | | | | | | | |
| Hippocampus | + | | + | + | + | | + | + |
| Thalamus | + | | + | | + | | + | |
| Superior | + | | + | + | + | | + | + |
| colliculus | | | | | | | | |
| Cerebellum | + | + | + | + | + | + | + | + |
| Spinal cord | + | + | + | + | + | + | + | + |

### Example 5: AAV Packaging Using Capsid X

To further understand the packaging capability of Capsid X, packaging at small scale, purification, and analytics were investigated. The various vectors used in this Example are outlined in Table 3.

TABLE 3
Capsid X Vectors

| Vector | Description |
|---|---|
| pHM-00228 | Capsid X Cap |
| scGFP | Self-Complementary GFP |
| pHM-00224 | Control (AAVHSC15) with wild type Rep |

### Seeding and Transfection

Two T-225 flasks were seeded at a density of 3.00E4 cells/cm² and cultured for 3 days at 37° C., with 5% CO₂, and 80% humidity using a HEK293 cell line. On day three, the T-225 flasks were transfected according to the experimental design set forth in Table 4 below.

TABLE 4
Experimental Design

| | | Vector Elements | | |
|---|---|---|---|---|
| Flask | Transfected Vectors | ITR | Rep | Cap |
| 1 | pHM-00224 + scGFP | AAV2 | WT-AAV2 | Control |
| 2 | pHM-00228 + scGFP | AAV2 | WT-AAV2 | Capsid X |

Harvest

72 hours after transfection, the cells in each T-flask were dislodged by agitation, collected in 50 mL conical tubes and centrifuged at 2000 RPM for 20 minutes. The supernatant was discarded, and the cell pellet was saved. Each flask was washed with 20 mL DPBS and then added to the pellets. The samples were then centrifuged at 2000 RPM for 15 minutes. After discarding the supernatant, the cells were resuspended in 1.9 mL of lysis buffer and incubated at 37° C. for 1 hour. They were then centrifuged at 4700 RPM for 20 minutes, the supernatant containing AAV now termed “crude lysate” was saved and the pellet discarded. Individual samples were taken for each flask: 25 μl for analytics, 25 μL for retain, and 100 μl saved to be run on a Western Blot. The crude lysates of each flask were transferred into individual conical tubes to be saved for batch purification.

### Batch Purification

1 mL of 50% slurry was made with AAV9 resin in 10% ethanol (EtOH) storage solution. The slurry was centrifuged at 2000×g for 3 min, the supernatant removed, and 0.5 mL of equilibration buffer was added to recreate a 50% slurry. This was repeated for 2 more washes to remove residual EtOH.

Each vector's crude lysate was thawed at room temperature. 1004, of the 50% slurry solution was added to each crude lysate. The resin was mixed with the crude lysate overnight at 4° C., with constant mixing by inversion.

The next day, the samples were transferred to a purification column (Amicon® Pro, MilliporeSigma). Any residual resin was collected using equilibration buffer and added to the purification column. Two additional wash steps were performed directly on the purification column, flow-through was removed as needed, and was sampled for analytics.

The purification columns were transferred to new conical tubes. 300 μL of elution buffer was added directly to the packed resin and resuspended twice, slowly by pipette. After 3 minutes of incubation, the purification columns were centrifuged at 2000×g for 1 minute. Neutralization buffer was added directly to the eluent at 1% of the total elution volume. Final batch purified products were moved to −80° C.

Results

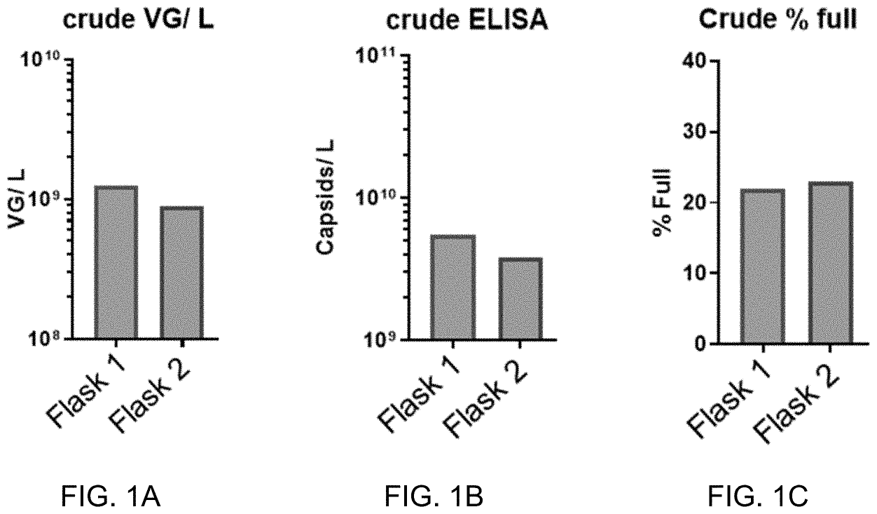

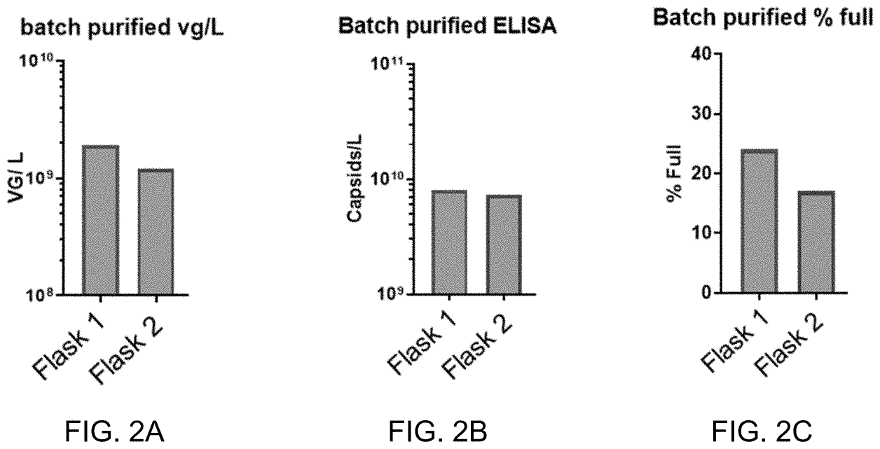

Capsid X productivity was tested in the crude lysates (FIGS. 1A, 1B, and 1C) as well as batch purified products (FIGS. 2A, 2B, and 2C) obtained as described above. Productivity titers were determined using digital droplet PCR (ddPCR) and ELISA. FIGS. 1A, 1B, and 1C are graphs showing Capsid X productivity titers in crude lysates as determined by ddPCR (FIG. 1A) and ELISA (FIG. 1B), and the percentage of full capsids detected, as determined using ELISA and ddPCR values (FIG. 1C). FIGS. 2A, 2B, and 2C are graphs showing Capsid X productivity titers in batch purified products as determined by ddPCR (FIG. 2A) and ELISA (FIG. 2B), and the percentage of full capsids detected, as determined using ELISA and ddPCR values (FIG. 2C). The experimental conditions in FIGS. 1A, 1B, 1C, 2A, 2B, and 2C are as described in Table 4.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. An AAV capsid protein comprising:
an amino acid sequence having at least 95% sequence identity with the amino acid sequence of amino acids 203-736 of SEQ ID NO:1, wherein the amino acid in the capsid protein corresponding to amino acid 412 of SEQ ID NO:1 is T;
an amino acid sequence having at least 95% sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; or the amino acid in the capsid protein corresponding to amino acid 412 of SEQ ID NO:1 is T; or
an amino acid sequence having at least 95% sequence identity with the amino acid sequence of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; or the amino acid in the capsid protein corresponding to amino acid 412 of SEQ ID NO:1 is T.

2. The AAV capsid protein of claim 1, wherein:
the AAV capsid protein comprises an amino acid sequence having at least 99% sequence identity with the amino acid sequence of amino acids 203-736 of SEQ ID NO:1;
the AAV capsid protein comprises the amino acid sequence of amino acids 203-736 of SEQ ID NO:1; or
the amino acid sequence of the AAV capsid protein consists of the amino acid sequence of amino acids 203-736 of SEQ ID NO:1.

3-5. (canceled)

6. The AAV capsid protein of claim 1, wherein:
the AAV capsid protein comprises an amino acid sequence having at least 99% sequence identity with the amino acid sequence of amino acids 138-736 of SEQ ID NO:1;
the AAV capsid protein comprises the amino acid sequence of amino acids 138-736 of SEQ ID NO:1; or
the amino acid sequence of the AAV capsid protein consists of the amino acid sequence of amino acids 138-736 of SEQ ID NO:1.

7-9. (canceled)

10. The AAV capsid protein of claim 1, wherein:
the AAV capsid protein comprises an amino acid sequence having at least 99% sequence identity with the amino acid sequence of SEQ ID NO: 1;
the AAV capsid protein comprises the amino acid sequence of SEQ ID NO:1; or
the amino acid sequence of the AAV capsid protein consists of the amino acid sequence of SEQ ID NO:1.

11-12. (canceled)

13. An isolated polynucleotide encoding the AAV capsid protein of claim 1.

14. A vector comprising the polynucleotide of claim 13, optionally wherein:
the vector is a plasmid;
the vector is an expression vector; and/or
the vector is a viral vector, optionally wherein the viral vector is a retrovirus vector, a herpes virus vector, a baculovirus vector, or an adenovirus vector.

15-17. (canceled)

18. A recombinant cell comprising:
a polynucleotide encoding an AAV capsid protein comprising:
an amino acid sequence having at least 95% sequence identity with the amino acid sequence of amino acids 203-736 of SEQ ID NO:1, wherein the amino acid in the capsid protein corresponding to amino acid 412 of SEQ ID NO:1 is T;
an amino acid sequence having at least 95% sequence identity with the amino acid sequence of amino acids 138-736 of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; or the amino acid in the capsid protein corresponding to amino acid 412 of SEQ ID NO:1 is T; or
an amino acid sequence having at least 95% sequence identity with the amino acid sequence of SEQ ID NO:1, wherein: the amino acid in the capsid protein corresponding to amino acid 146 of SEQ ID NO:1 is I; the amino acid in the capsid protein corresponding to amino acid 157 of SEQ ID NO:1 is V; or the amino acid in the capsid protein corresponding to amino acid 412 of SEQ ID NO:1 is T; or
the vector of claim 14.

19. A method of producing an AAV capsid protein, the method comprising culturing the recombinant cell of claim 18 under conditions whereby the polynucleotide is expressed and the capsid is produced.

20. A recombinant adeno-associated virus (rAAV) comprising:
(a) a capsid comprising the AAV capsid protein of claim 1; and
(b) an rAAV genome, optionally comprising a transgene or editing genome, optionally wherein:
the transgene encodes a polypeptide;
the transgene encodes an miRNA, shRNA, siRNA, antisense RNA, gRNA, antagomir, miRNA sponge, RNA aptazyme, RNA aptamer, lncRNA, ribozyme or mRNA, and/or
the transgene is operably linked to a transcriptional regulatory element.

21-25. (canceled)

26. A method for transducing a cell, the method comprising contacting the cell with the rAAV of claim 20 under conditions whereby the cell is transduced, optionally wherein:
the cell is a blood, liver, heart, joint tissue, muscle, brain, kidney, or lung cell;
the cell is a cell of the central nervous system or a cell of the peripheral nervous system;
the method is performed ex vivo or in vitro; and/or
the cell is in a subject and the rAAV is administered to the subject, optionally wherein the rAAV is administered to the subject intravenously, intraperitoneally, subcutaneously, intramuscularly, intrathecally, or intradermally; and/or the subject is human.

27. A method for expressing a transgene in a cell, the method comprising contacting the cell with the rAAV of claim 20 under conditions whereby the cell is transduced and the transgene is expressed, optionally wherein:
the cell is a blood, liver, heart, joint tissue, muscle, brain, kidney, or lung cell;
the cell is a cell of the central nervous system or a cell of the peripheral nervous system;
the method is performed ex vivo or in vitro; and/or
the cell is in a subject and the rAAV is administered to the subject, optionally wherein the rAAV is administered to the subject intravenously, intraperitoneally, subcutaneously, intramuscularly, intrathecally, or intradermally; and/or the subject is human.

28. A method for editing a target locus in a genome of a cell, the method comprising contacting the cell with the rAAV of claim 20 under conditions whereby the cell is transduced and the target locus is edited, optionally wherein:
the cell is a blood, liver, heart, joint tissue, muscle, brain, kidney, or lung cell;
the cell is a cell of the central nervous system or a cell of the peripheral nervous system;
the method is performed ex vivo or in vitro; and/or
the cell is in a subject and the rAAV is administered to the subject, optionally wherein the rAAV is administered to the subject intravenously, intraperitoneally, subcutaneously, intramuscularly, intrathecally, or intradermally; and/or the subject is human.

29-34. (canceled)

35. A packaging system for preparation of an rAAV, wherein the packaging system comprises:
(a) a first nucleotide sequence encoding one or more AAV Rep proteins;
(b) a second nucleotide sequence encoding the AAV capsid protein of claim 1; and
(c) a third nucleotide sequence comprising an rAAV genome sequence,
optionally wherein the packaging system comprises a first vector comprising the first nucleotide sequence and the second nucleotide sequence, and a second vector comprising the third nucleotide sequence.

36. (canceled)

37. The packaging system of claim 35, further comprising a fourth nucleotide sequence comprising one or more helper virus genes, optionally wherein:
the fourth nucleotide sequence is comprised within a third vector;
the fourth nucleotide sequence comprises one or more genes from a virus selected from the group consisting of adenovirus, herpesvirus, vaccinia virus, and cytomegalovirus (CMV); and/or
the first vector, the second vector, and/or the third vector is a plasmid.

38-40. (canceled)

41. A method for recombinant preparation of an rAAV, the method comprising introducing the packaging system of claim 35 into a cell under conditions whereby the rAAV is produced.

42. (canceled)


SEQUENCE LISTING (23 sequences)

SEQ ID NO: 1 - protein, 736 aa, Artificial Sequence
  Synthetic amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPIEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 365 bp, Artificial Sequence
  Synthetic nucleic acid sequence
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 3 - DNA, 380 bp, Artificial Sequence
  Synthetic nucleic acid sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 4 - DNA, 479 bp, Artificial Sequence
  Synthetic nucleic acid sequence
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggag

SEQ ID NO: 5 - DNA, 1246 bp, Artificial Sequence
  Synthetic nucleic acid sequence
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 6 - DNA, 953 bp, Artificial Sequence
  Synthetic nucleic acid sequence
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 7 - DNA, 1168 bp, Artificial Sequence
  Synthetic nucleic acid sequence
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 92 bp, Artificial Sequence
  Synthetic nucleic acid sequence
aagaggtaagggtttaagggatggttggttggtggggtattaatgtttaattacctggagcacctgcctgaaatcactttttttcaggttgg

SEQ ID NO: 9 - DNA, 79 bp, Artificial Sequence
  Synthetic nucleic acid sequence
aagaggtaagggtttaagggatggttggttggtggggtattaatgtttaattacctggagcacctgcctgaaatcactt

SEQ ID NO: 10 - DNA, 95 bp, Artificial Sequence
  Synthetic nucleic acid sequence
cctctgctaaccatgttcatgccttcttctttttcctacagctcctgggcaacgtgctggttattgtgctgtctcatcattttggcaaagaattc

SEQ ID NO: 11 - DNA, 90 bp, Artificial Sequence
  Synthetic nucleic acid sequence
cctctgctaaccatgttcatgccttcttctttttcctacagctcctgggcaacgtgctggttattgtgctgtctcatcattttggcaaag

SEQ ID NO: 12 - DNA, 918 bp, Artificial Sequence
  Synthetic nucleic acid sequence
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagag

SEQ ID NO: 13 - DNA, 192 bp, Artificial Sequence
  Synthetic nucleic acid sequence
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 14 - DNA, 205 bp, Artificial Sequence
  Synthetic nucleic acid sequence
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 15 - DNA, 913 bp, Artificial Sequence
  Synthetic nucleic acid sequence
tagggaggtcctgcacagaaggggaggagggggcagcagctgtctgaccactgttggtcttgcaacttgtgtccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggattgtaactgaaatgcttcactggtgctccttttgttttaaggcattggatcttcatagctactgatcgtgcccaagcacacagtatctgcagcaaccacttaggcctccaggaatgtggtgaccattgaccctaattcattccccttcatggatcctatgtaaccatcctccaaaaagagctttcgcaaactcaaataaacacaggaaaggaagaccttcttatctttgagagtatatgtttagccctatagctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 16 - DNA, 423 bp, Artificial Sequence
  Synthetic nucleic acid sequence
gctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 17 - DNA, 170 bp, Artificial Sequence
  Synthetic nucleic acid sequence
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 18 - DNA, 1873 bp, Artificial Sequence
  Synthetic nucleic acid sequence
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 19 - DNA, 1061 bp, Artificial Sequence
  Synthetic nucleic acid sequence
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 20 - DNA, 398 bp, Artificial Sequence
  Synthetic nucleic acid sequence
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 21 - protein, 621 aa, Artificial Sequence
  Synthetic amino acid sequence
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 22 - DNA, 192 bp, Artificial Sequence; identical to SEQ ID NO: 13
  Synthetic nucleic acid sequence

SEQ ID NO: 23 - DNA, 72 bp, Artificial Sequence
  Synthetic nucleic acid sequence
gggggaggctgctggtgaatattaaccaaggtcaccccagttatcggaggagcaaacaggggctaagtccac